(19) United States
(12) Patent Application Publication
(10) Pub. No.: US 2024/0287616 A1
(43) Pub. Date: Aug. 29, 2024

(54) METHODS OF DETECTING NTRK FUSION PROTEINS

(71) Applicant: Strata Oncology, Inc., Ann Arbor, MI (US)

(72) Inventors: Daniel Reed Rhodes, Chelsea, MI (US); Scott Arthur Tomlins, Ann Arbor, MI (US); David Bryan Johnson, Chelsea, MI (US)

(21) Appl. No.: 18/571,643

(22) PCT Filed: Jun. 21, 2022
(86) PCT No.: PCT/US22/34417

§ 371 Date: Dec. 18, 2023

Related U.S. Application Data
(60) Provisional application No. 63/212,577, filed on Jun. 18, 2021.

(51) Int. Cl. C12Q 1/6886

(52) U.S. Cl. CPC C12Q 1/6886; C12Q 2600/106; C12Q 2600/158; G01N 2800/52

(57) ABSTRACT

Disclosed herein are methods of detecting differential over-expression of 3′ exons relative to 5′ exons of the Neurotrophic Tyrosine Receptor Kinase (NTRK) gene to facilitate the identification of subjects having NTRK fusion proteins. Additionally, methods of treating cancer by administering NTRK inhibitors to a subject identified as having tumors which express NTRK fusion protein, are also disclosed.

### RELATED APPLICATION

This application claims priority to, and the benefit of, co-pending U.S. Provisional No. 63/212,577, filed Jun. 18, 2021. The disclosure of said provisional application is hereby incorporated by reference in its entirety.

### BACKGROUND OF THE INVENTION

NTRK gene fusions involving either NTRK1, NTRK2, or NTRK3 are oncogenic drivers of various adult and pediatric tumor types, and targeted therapies suitable for treatment of cancers expressing NTRK gene fusions are available. Current methods of NTRK fusion detection rely upon specific detection of particular fusion proteins. However, a need remains for methods of detecting the presence of NTRK fusions, especially NTRK fusions that are less common.

### SUMMARY OF THE INVENTION

Some aspects of the present disclosure are directed to a method of treatment, comprising: (a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from a subject; (b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene; (c) determining that the tumor expresses an NTRK fusion protein when (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4; and (d) administering an NTRK inhibitor to the tumor identified as expressing an NTRK fusion protein. In some embodiments, the NTRK gene is selected from NTRK1, NTRK2, and NTRK3.

In some embodiments, the expression level of the RNA transcripts of the NTRK gene is determined by measuring the expression of RNA transcripts of a portion of the NTRK gene comprising the junction of exons 16-17. In some embodiments, measuring an expression level of a 3′ exon portion of the NTRK gene comprises measuring the expression of RNA transcripts of one or more portions of the NTRK gene comprising the junction of exons 14-15, 15-16, 16-17, and/or 17-18. In some embodiments, measuring an expression level of a 5′ exon portion of the NTRK gene comprises measuring the expression of RNA transcripts of one or more portions of the NTRK gene comprising the junction of exons 2-3, 3-4, and/or 4-5.

In some embodiments, the at least one reference gene comprises at least one gene selected from LRP1, MRPL13, TBP, HMBS, ITGB7, MYC, CIAO1, CTCF, EIF2B1, GGNBP2, and SLC4A1AP.

In some embodiments, the tumor specimen is a formalin-fixed paraffin-embedded (FFPE) tumor specimen.

In some embodiments, the expression levels of RNA transcripts are measured using PCR and next-generation sequencing (NGS).

In some embodiments, the method further comprises measuring an expression level of RNA transcripts for a fusion protein in the biological sample obtained from the tumor specimen by PCR amplification of the fusion protein breakpoint. In some embodiments, the fusion protein is an NTRK, ALK, ROS1, or RET fusion protein.

In some embodiments, the method further comprises determining if the tumor will be responsive to a checkpoint inhibitor therapy by measuring the expression levels of RNA transcripts of one or more genes in the biological sample obtained from the tumor specimen found to be correlated with efficacy of the checkpoint inhibitor therapy. In some embodiments, the method further comprises determining if the tumor will be responsive to an anti-cancer therapy by measuring the expression levels of RNA transcripts of one or more gene mutations in the biological sample obtained from the tumor specimen associated with efficacy for the anti-cancer therapy. In some embodiments, the method further comprises determining the tumor mutation burden and/or the microsatellite instability in the biological sample obtained from the tumor specimen. In some embodiments, the NTRK inhibitor is one or more agents selected from the group consisting of AG-879, AZ-23, AZD-1480, Belizatinib (TSR011), BMS-754807, Crizotinib, Entrectinib, Forctinib, GW-2580, K252a, Larotrectinib, Midostaurin (PKC412), and PLX7486.

Some aspects of the present disclosure are directed to a method of identifying a subject who would benefit from NTRK inhibitor therapy, comprising (a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject; (b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene; (c) determining that the tumor expresses an NTRK fusion protein when (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4; and (d) providing a determination if the subject has a tumor expressing an NTRK fusion protein and therefore would benefit from NTRK inhibitor therapy.

Some aspects of the present disclosure are directed to a method of treatment of a subject in need thereof with a NTRK inhibitor therapy, comprising administering to said subject the NTRK inhibitor therapy, wherein said subject in need thereof was previously identified by a method comprising: (a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject; (b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene; and (c) determining that the tumor expresses an NTRK fusion protein when (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a graph showing NTRK1 imbalance versus NTRK1 fusions called by methods described herein.

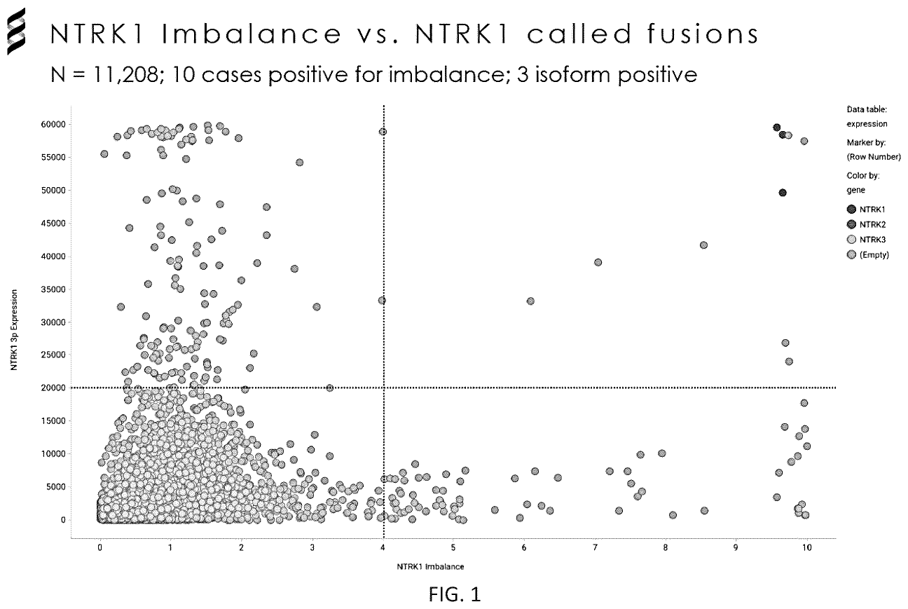

FIG. 2 is a graph showing NTRK3 imbalance versus NTRK3 fusions called by methods described herein.

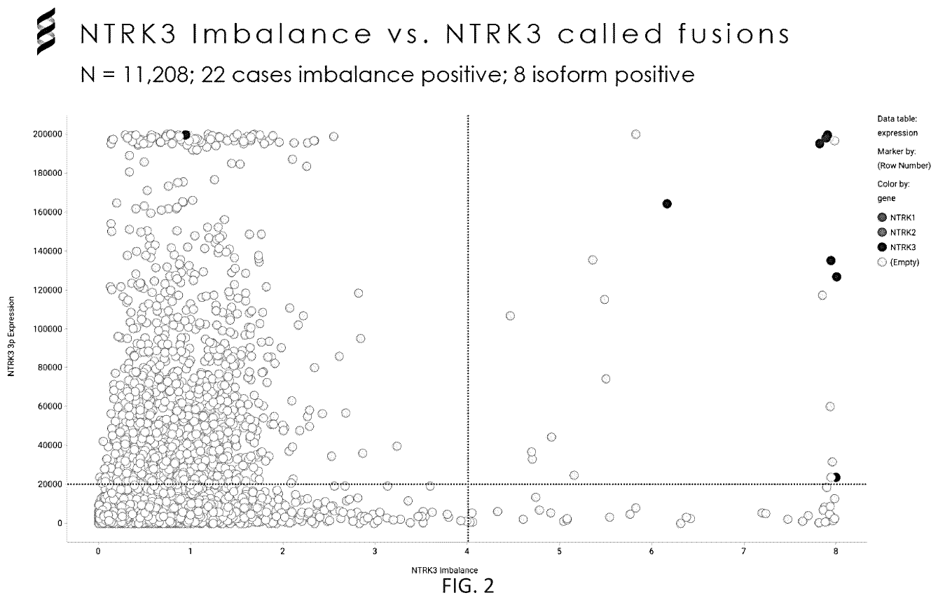

FIG. 3 is a graph showing NTRK1 balance validation via sequencing of a fusion isoform-negative, imbalance positive case.

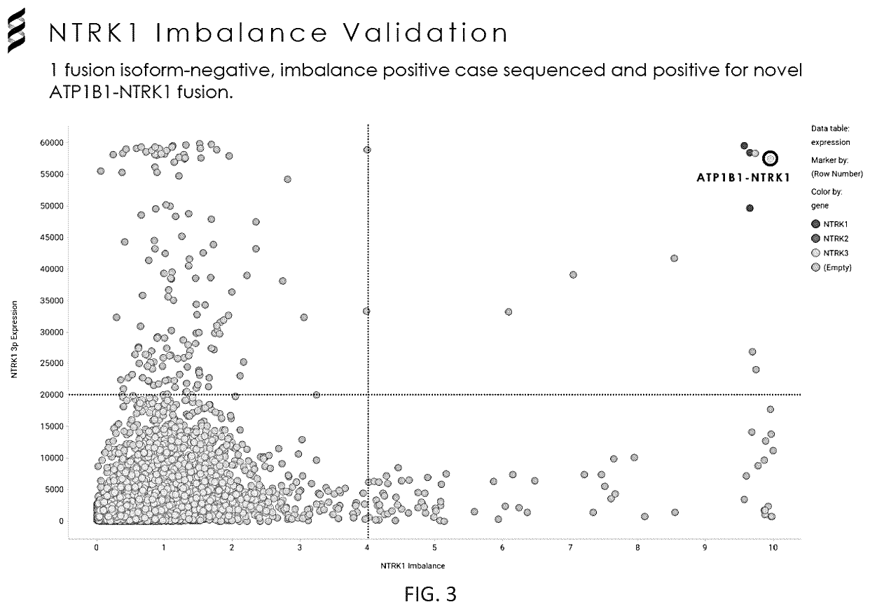

FIG. 4 is a graph showing NTRK3 balance validation via sequencing of 2 fusion isoform-negative, imbalance positive cases.

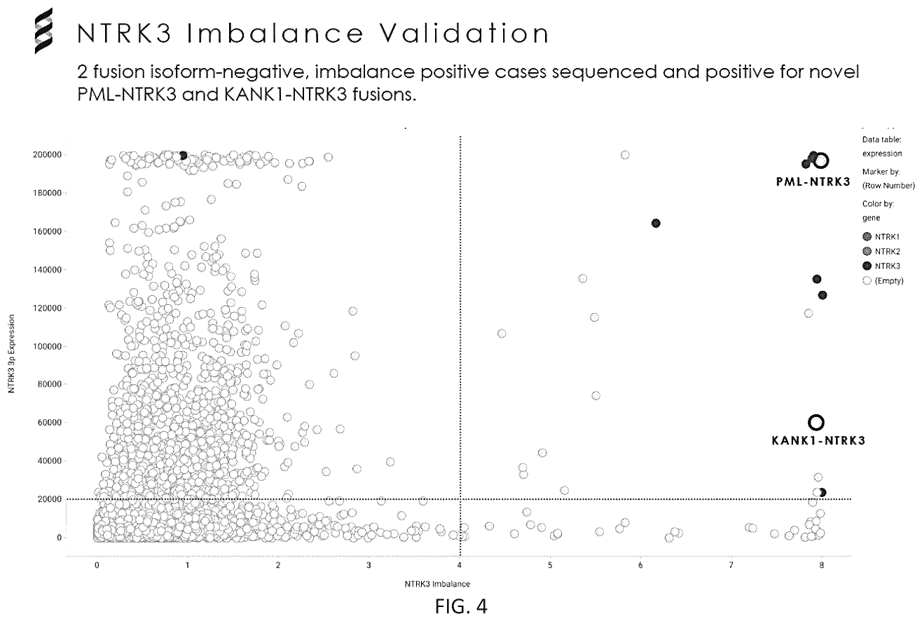

FIG. 5 depicts sequence read information for ROS1 p.G2032R.

FIG. 6 depicts the expression levels of several housekeeping genes as well as expression levels of multiple oncogenes. FIG. 6 demonstrates high ROS1 3′ exon expression, which when taken together with the lower ROS1 5′ exon expression, demonstrates a 3′/5′ exon expression mismatch indicative of the presence of a ROS1 fusion protein.

### DETAILED DESCRIPTION OF THE INVENTION

Disclosed herein are methods of detecting differential over-expression of 3′ exons relative to 5′ exons that can identify subjects having NTRK fusion proteins even when such fusion proteins are uncommon NTRK fusions that are not detected by PCR-based amplifications taught in the art. Such methods enable identification and treatment of appropriate patients with NTRK inhibitors without the need to screen patient samples for specific NTRK fusions.

Some aspects of the present disclosure are directed to a method of treatment, comprising:
(a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from a subject;
(b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene;
(c) determining that the tumor expresses an NTRK fusion protein when (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4; and (optionally)
(d) administering an NTRK inhibitor to the tumor identified as expressing an NTRK fusion protein.

The tropomyosin receptor kinase (Trk or TRK) family of tyrosine kinase receptors are multi-domain transmembrane proteins that play an important role in a wide spectrum of neuronal responses including survival, differentiation, growth, and regeneration. The Trk receptors are expressed abundantly in the nervous system, as well as in many other non-neuronal cell types and tissues, including monocytes, the lung, bone, and pancreatic beta cells. There are three members of the Trk family: TrkA, TrkB, and TrkC, encoded by the NTRK1, NTRK2, and NTRK3 genes respectively. TrkA, TrkB, and TrkC are characterized as high affinity receptors for naturally-occurring neurotrophins, a family of protein growth factors which includes nerve growth factor (NGF), brain derived neurotrophic factor (BDNF), neurotrophin-3 (NT-3), and neurotrophins-4/5 (NT-4/5). Mature neurotrophins bind a selective Trk receptor with relatively high affinity (e.g., TrkB-BDNF, TrkA-NGF, and TrkC-NT-3), resulting in the activation of intracellular tyrosine kinase signaling cascades (e.g., SHC-RAS-MAPK, PI3K-AKT, or PLCy-PKC) that mediate neurotrophin function (e.g., neuronal growth and survival).

In some embodiments, the NTRK gene is selected from NTRK1 (e.g., NCBI Entrez Gene: 4914), NTRK2 (e.g., NCBI Entrez Gene: 4915), and NTRK3 (e.g., NCBI Entrez Gene: 4916).

In some embodiments of the methods disclosed herein, the expression levels of RNA transcripts are measured using PCR and next-generation sequencing. Embodiments of the methods disclosed herein preferably apply, include, and/or are otherwise associated with next-generation sequencing (NGS) (e.g., processing biological samples to generate sequence libraries for sequencing with next-generation sequencing systems; etc.). Embodiments of the methods disclosed herein can include, apply, and/or otherwise be associated with semiconductor-based sequencing technologies. Additionally or alternatively, embodiments of the methods disclosed herein can include, apply, and/or otherwise be associated with any suitable sequencing technologies (e.g., sequencing library preparation technologies; sequencing systems; sequencing output analysis technologies; etc.). Sequencing technologies preferably include next-generation sequencing technologies. Next-generation sequencing technologies can include any one or more of high-throughput sequencing (e.g., facilitated through high-throughput sequencing technologies; massively parallel signature sequencing, Polony sequencing, 454 pyrosequencing, Illumina sequencing, SOLID sequencing, Ion Torrent semiconductor sequencing and/or other suitable semiconductor-based sequencing technologies, DNA nanoball sequencing, Heliscope single molecule sequencing. Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), sequencing-by-synthesis, tunneling currents sequencing, sequencing by hybridization, mass spectrometry sequencing, microscopy-based techniques, and/or any suitable next-generation sequencing technologies.

Additionally or alternatively, sequencing technologies can include any one or more of: capillary sequencing. Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

Embodiments of the methods disclosed herein can include, apply, perform, and/or otherwise be associated with any one or more of: sequencing operations, alignment operation (e.g., sequencing read alignment; etc.), lysing operations, cutting operations, tagging operations (e.g., with barcodes; etc.), ligation operations, fragmentation operations, amplification operations (e.g., helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), etc.), purification operations, cleaning operations, suitable operations for sequencing library preparation, suitable operations for facilitating sequencing and/or downstream analysis, suitable sample processing operations, and/or any suitable sample- and/or sequence-related operations.

Any suitable portion of NTRK RNA (e.g., NTRK1, NTRK2, or NTRK3) may be sequenced to determine expression of the NTRK. In some embodiments, the expression level of the RNA transcripts of the NTRK gene is determined by measuring the expression of RNA transcripts of a portion of the NTRK gene RNA (e.g., NTRK1, NTRK2, or NTRK3) comprising the junction of exons 16-17.

Any suitable portion of NTRK RNA (e.g., NTRK1, NTRK2, or NTRK3) located in the 3′ exon portion of the RNA may be sequenced to determine expression of the NTRK 3′ exon portion. In some embodiments, measuring an expression level of a 3′ exon portion of the NTRK gene comprises measuring the expression of RNA transcripts of one or more portions of the NTRK gene comprising the junction of exons 14-15, 15-16, 16-17, and/or 17-18.

Any suitable portion of NTRK RNA (e.g., NTRK1, NTRK2, or NTRK3) located in the 5′ exon portion of the RNA may be sequenced to determine expression of the NTRK 5′ exon portion. In some embodiments, measuring an expression level of a 5′ exon portion of the NTRK gene further comprises measuring the expression of RNA transcripts of one or more portions of the NTRK gene comprising the junction of exons 2-3, 3-4, and/or 4-5.

As used herein, a reference gene is a gene that varies little in its median expression among relevant different biological samples. The reference gene is not limited and may be any suitable reference gene. In some embodiments, the at least one reference gene comprise at least one gene selected from LRP1, MRPL13, TBP, HMBS, ITGB7, MYC, CIAO1, CTCF, EIF2B1, GGNBP2, and SLC4A1AP. In some embodiments, at least two, three, four, five, six, seven, eight, nine, ten, or eleven reference genes (e.g., reference genes selected from LRP1, MRPL13, TBP, HMBS, ITGB7, MYC, CIAO1, CTCF, EIF2B1, GGNBP2, and SLC4A1AP) are measured.

In some embodiments, an NTRK fusion protein is detected when the ratio of the normalized expression level of the RNA transcripts of the 3′ exon portion of the NTRK gene to the normalized expression level of the RNA transcripts of the 5′ exon portion of the NTRK gene is greater than 4, 4.1, 4.2, 4.3, 4.4, 4.5, 4.6, 4.7, 4.8, 4.9, 5, 5.5, 6, or more.

The tumor sample may be from any cancer and is not limited. As used herein, the term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypercosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid cancer. In some embodiments, the cancer is breast, cholangiocarcinoma, colorectal, gynecological, neuroendocrine, non-small cell lung, salivary gland, pancreatic, sarcoma or thyroid cancer.

In some embodiments, the tumor specimen has undergone fixation. The method of fixation is not limited and may be any method of fixation known in the art. In some embodiments, the tumor specimen is a formalin-fixed paraffin-embedded (FFPE) tumor specimen.

In some embodiments, the tumor specimen comprises less than about 25 nanograms (ng) of genomic material. In some embodiments, the tumor specimen comprises less than about 20 ng of genomic material. In some embodiments, the tumor specimen comprises less than about 15 ng of genomic material. In some embodiments, the tumor specimen comprises less than about 12 ng of genomic material. In some embodiments, the tumor specimen comprises less than 10 ng of genomic material. In some embodiments, the tumor specimen comprises less than 7.5 ng of genomic material. In some embodiments, the tumor specimen comprises less than 5 ng of genomic material.

The area of the tumor sample (mm²) is not limited. In some embodiments, the area of the tumor sample is less than the area needed for variant calling methods used in the art. In some embodiments, the tumor sample is a sample having an area of less than 5 mm². In some embodiments, the tumor sample is a sample having an area of less than 10 mm². In some embodiments, the tumor sample is a sample having an area of less than 15 mm². In some embodiments, the tumor sample is a sample having an area of less than 20 mm². In some embodiments, the tumor sample is a sample having an area of less than 25 mm². In some embodiments, the tumor sample is a sample having an area of less than 30 mm². In some embodiments, the tumor sample is a sample having an area of less than 35 mm². In some embodiments, the tumor sample is a sample having an area of less than 40 mm².

In some embodiments, the method further comprises measuring an expression level of RNA transcripts for a fusion protein in the biological sample obtained from the tumor specimen by PCR amplification of the fusion protein breakpoint. In some embodiments, the fusion protein is an NTRK, ALK, ROS1, or RET fusion protein. In some embodiments, the NTRK fusion protein is CD74-NTRK1, LMNA-NTRK1, MPRIP-NTRK1, TPM3-NTRK1, SQSTM1-NTRK1, PPL-NTRK1, AFAP1-NTRK2, PAN3-NTRK2, TRIM24-NTRK2, BTBD1-NTRK3, or ETV6-NTRK3. In some embodiments, the ALK fusion protein is TPM3-ALK, TPM4-ALK, ATIC-ALK, CLTC-ALK, RanBP2-ALK. TFGL/S-ALK, CARS-ALK, or MSN-ALK. In some embodiments, the ROS1 fusion protein is CD74-ROS1, SLC34A2-ROS1, TPM3-ROS1, SDC4-ROS1, EZR-ROS1, LRIG-ROS1, KDELR2-ROS1, CCDC6-ROS1, FIG-ROS1, TPD52L1-ROS1, CEP85L-ROS1, ZCCHC8-ROS1, CCDC30-ROS1, TFG-ROS1, TMEM106B-ROS1, YWHAE-ROS1, MSN-ROS1, PWWP2A-ROS1, FYN-ROS1, MKX-ROS1, PPFIBP1-ROS1, ERC1-ROS1, MY05A-ROS1, CLIP1-ROS1, HLA-A-ROS1, KIAA1598-ROS1, CLTC-ROS1, LIMA1-ROS1, NFkB2-ROS1, NCOR2-ROS1, KCL1-ROS1, or TBL1XR1-ROS1. In some embodiments, the RET fusion protein is ACBD5-RET, AFAPIL2-RET, AKAP13-RET, ANKRD26-RET, BCR-RET, CDC123-RET, CCDC6-RET, CLIP2-RET, CUX1-RET, DLG5-RET, EPHA5-RET, ERC1-RET, ETL4-RET, ETV6-RET, FGFR1OP-RET, FKBP15-RET, FRMD4A-RET, GEMIN5-RET, GOLGA5-RET, HOOK3-RET, KHDRBS1-RET, KIAA1468-RET, KIF13A-RET, KIF5B-RET, KTN1-RET, MYH10-RET, MYH13-RET, OR MYO5A-RET.

In some embodiments, the method further comprises determining if the tumor will be responsive to a checkpoint inhibitor therapy by measuring the expression levels of RNA transcripts of one or more genes in the biological sample obtained from the tumor specimen found to be correlated with efficacy of the checkpoint inhibitor therapy. In some embodiments, the expression of one or more of the RNA transcripts of PD-L1, PD-L2, CD8A, TOP2A, and GZMA are measured.

In some embodiments, the method further comprises determining if the tumor will be responsive to an anti-cancer therapy by measuring the expression levels of RNA transcripts of one or more gene mutations in the biological sample obtained from the tumor specimen associated with efficacy for the anti-cancer therapy. In some embodiments, the gene mutation is a BRAF, KIT, NF, NRAS, or PTEN mutation.

In some embodiments, the method further comprises determining the tumor mutation burden and/or the microsatellite instability in the biological sample obtained from the tumor specimen. Methods of detecting mutations (e.g., TMB) are not limited. In some embodiments, mutations are detected, calculated or obtained via NGS. In some embodiments, TMB includes non-coding (at highly characterized genomic loci) and coding, synonymous and non-synonymous, single and multi-nucleotide (two bases) variants present at >10% variant allele frequency (VAF). In some embodiments, mutations per megabase (Mb) estimates and associated 90% confidence interval are calculated via the total number of positions with sufficient depth of coverage necessary for definitive assessment (maximum possible 1.7 Mb).

In some embodiments, the NTRK inhibitor is one or more selected from the group consisting of AG-879, AZ-23, AZD-1480, Belizatinib (TSR011), BMS-754807, Crizotinib, Entrectinib, Foretinib, GW-2580, K252a, Larotrectinib, Midostaurin (PKC412), and PLX7486.

As used herein, a “subject” is a mammal, including but not limited to a primate (e.g., a human), rodent (e.g., mouse or rat) dog, cat, horse, cow, pig, sheep, goat, or chicken. Preferred subjects are human subjects. The human subject may be a pediatric or adult subject. In some embodiments, the subject is at least about 40 years old, at least about 45 years old, at least about 50 years old, at least about 55 years old, at least about 60 years old, at least about 65 years old, at least about 70 years old, at least about 75 years old, at least about 80 years old, at least about 85 years old, or at least about 90 years old. In some embodiments, the subject is less than 20 years old, 18 years old, 15 years old, 10 years old, 5 years old, or 2 years old. In some embodiments, the subject has been diagnosed with, is suspected of having, or is at risk of having a tumor described herein.

Some aspects of the present disclosure are directed to a method of identifying a subject who would benefit from NTRK inhibitor therapy, comprising
(a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject;
(b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene;
(c) determining that the tumor expresses an NTRK fusion protein when (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4; and
(d) providing a determination of whether the subject has a tumor expressing an NTRK fusion protein and therefore would benefit from NTRK inhibitor therapy. In appropriate cases in which the subject has a tumor expressing an NTRK fusion protein the subject can, optionally, then be treated with NTRK inhibitor therapy.

The subject is not limited and may be any subject disclosed herein. In some embodiments, the subject is a human (e.g., adult human or juvenile human). In some embodiments, the NTRK gene is selected from NTRK1 (e.g., NCBI Entrez Gene: 4914), NTRK2 (e.g., NCBI Entrez Gene: 4915), and NTRK3 (e.g., NCBI Entrez Gene: 4916). In some embodiments of the methods disclosed herein, the expression levels of RNA transcripts are measured using PCR and next-generation sequencing.

Any suitable portion of NTRK RNA (e.g., NTRK1, NTRK2, or NTRK3) may be sequenced to determine expression of the NTRK. In some embodiments, the expression level of the RNA transcripts of the NTRK gene is determined by measuring the expression of RNA transcripts of a portion of the NTRK gene RNA (e.g., NTRK1, NTRK2, or NTRK3) comprising the junction of exons 16-17.

Any suitable portion of NTRK RNA (e.g., NTRK1, NTRK2, or NTRK3) located in the 3′ exon portion of the RNA may be sequenced to determine expression of the NTRK 3′ exon portion. In some embodiments, measuring an expression level of a 3′ exon portion of the NTRK gene comprises measuring the expression of RNA transcripts of one or more portions of the NTRK gene comprising the junction of exons 14-15, 15-16, 16-17, and/or 17-18.

Any suitable portion of NTRK RNA (e.g., NTRK1, NTRK2, or NTRK3) located in the 5′ exon portion of the RNA may be sequenced to determine expression of the NTRK 5′ exon portion. In some embodiments, measuring an expression level of a 5′ exon portion of the NTRK gene further comprises measuring the expression of RNA transcripts of one or more portions of the NTRK gene comprising the junction of exons 2-3, 3-4, and/or 4-5.

The reference gene is not limited and may be any suitable reference gene. In some embodiments, the at least one reference gene comprise at least one gene selected from LRP1, MRPL13, TBP. HMBS, ITGB7, MYC, CIAO1, CTCF, EIF2B1, GGNBP2, and SLC4A1AP.

In some embodiments, an NTRK fusion protein is detected when the ratio of the normalized expression level of the RNA transcripts of the 3′ exon portion of the NTRK gene to the normalized expression level of the RNA transcripts of the 5′ exon portion of the NTRK gene is greater than 4, 4.1, 4.2, 4.3, 4.4, 4.5, 4.6, 4.7, 4.8, 4.9, 5, 5.5, 6, or more.

The tumor sample may be from any cancer disclosed herein and is not limited. In some embodiments, the cancer is breast, cholangiocarcinoma, colorectal, gynecological, neuroendocrine, non-small cell lung, salivary gland, pancreatic, sarcoma or thyroid cancer.

In some embodiments, the tumor specimen has undergone fixation. The method of fixation is not limited and may be any method of fixation known in the art. In some embodiments, the tumor specimen is a formalin-fixed paraffin-embedded (FFPE) tumor specimen. The amount of genomic material in the tumor sample is not limited and may be any amount of genomic material disclosed herein. The area of the tumor sample is not limited and may be any area disclosed herein.

In some embodiments, the method further comprises measuring an expression level of RNA transcripts for a fusion protein in the biological sample obtained from the tumor specimen by PCR amplification of the fusion protein breakpoint. In some embodiments, the fusion protein is an NTRK, ALK, ROS1, or RET fusion protein (e.g., as disclosed herein).

In some embodiments, the method further comprises determining if the tumor will be responsive to a checkpoint inhibitor therapy by measuring the expression levels of RNA transcripts of one or more genes in the biological sample obtained from the tumor specimen found to be correlated with efficacy of the checkpoint inhibitor therapy. In some embodiments, the expression of one or more of the RNA transcripts of PD-L1, PD-L2, CD8A, TOP2A, and GZMA are measured.

In some embodiments, the method further comprises determining if the tumor will be responsive to an anti-cancer therapy by measuring the expression levels of RNA transcripts of one or more gene mutations in the biological sample obtained from the tumor specimen associated with efficacy for the anti-cancer therapy.

In some embodiments, the method further comprises determining the tumor mutation burden and/or the microsatellite instability in the biological sample obtained from the tumor specimen.

In some embodiments, the NTRK inhibitor is one or more selected from the group consisting of AG-879, AZ-23, AZD-1480, Belizatinib (TSR011), BMS-754807, Crizotinib, Entrectinib, Foretinib, GW-2580, K252a, Larotrectinib, Midostaurin (PKC412), and PLX7486.

Some aspects of the present disclosure are directed to a method of treatment of a subject in need thereof with a NTRK inhibitor therapy, comprising administering to said subject the NTRK inhibitor therapy, wherein said subject in need thereof was previously identified by a method comprising:
(a) measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject;
(b) normalizing the measured expression levels of the RNA transcripts of the NTRK gene, the 3′ exon portion of the NTRK gene, and the 5′ exon portion of the NTRK gene to the expression level of the RNA transcripts of the at least one reference gene; and
(c) determining that the tumor expresses an NTRK fusion protein when
   (i) the normalized expression level of the RNA transcripts of the NTRK gene is greater than a baseline expression level of RNA transcripts of the NTRK gene, and (ii) the ratio of the normalized expression level of the RNA transcripts of the 3′ exon portion of the NTRK gene to the normalized expression level of the RNA transcripts of the 5′ exon portion of the NTRK gene is greater than 4.

The subject is not limited and may be any subject disclosed herein. In some embodiments, the subject is a human (e.g., adult human or juvenile human). In some embodiments, the NTRK gene is selected from NTRK1 (e.g., NCBI Entrez Gene: 4914), NTRK2 (e.g., NCBI Entrez Gene: 4915), and NTRK3 (e.g., NCBI Entrez Gene: 4916). In some embodiments of the methods disclosed herein, the expression levels of RNA transcripts are measured using PCR and next-generation sequencing.

Any suitable portion of NTRK RNA (e.g., NTRK1, NTRK2, or NTRK3) may be sequenced to determine expression of the NTRK. In some embodiments, the expression level of the RNA transcripts of the NTRK gene is determined by measuring the expression of RNA transcripts of a portion of the NTRK gene RNA (e.g., NTRK1, NTRK2, or NTRK3) comprising the junction of exons 16-17.

Any suitable portion of NTRK RNA (e.g., NTRK1, NTRK2, or NTRK3) located in the 3′ exon portion of the RNA may be sequenced to determine expression of the NTRK 3′ exon portion. In some embodiments, measuring an expression level of a 3′ exon portion of the NTRK gene comprises measuring the expression of RNA transcripts of one or more portions of the NTRK gene comprising the junction of exons 14-15, 15-16, 16-17, and/or 17-18.

Any suitable portion of NTRK RNA (e.g., NTRK1, NTRK2, or NTRK3) located in the 5′ exon portion of the RNA may be sequenced to determine expression of the NTRK 5′ exon portion. In some embodiments, measuring an expression level of a 5′ exon portion of the NTRK gene further comprises measuring the expression of RNA transcripts of one or more portions of the NTRK gene comprising the junction of exons 2-3, 3-4, and/or 4-5.

The reference gene is not limited and may be any suitable reference gene. In some embodiments, the at least one reference gene comprise at least one gene selected from LRP1, MRPL13, TBP, HMBS, ITGB7, MYC, CIAO1, CTCF, EIF2B1, GGNBP2, and SLC4A1AP.

In some embodiments, an NTRK fusion protein is detected when the ratio of the normalized expression level of the RNA transcripts of the 3′ exon portion of the NTRK gene to the normalized expression level of the RNA transcripts of the 5′ exon portion of the NTRK gene is greater than 4, 4.1, 4.2, 4.3, 4.4, 4.5, 4.6, 4.7, 4.8, 4.9, 5, 5.5, 6, or more.

The tumor sample may be from any cancer disclosed herein and is not limited. In some embodiments, the cancer is breast, cholangiocarcinoma, colorectal, gynecological, neuroendocrine, non-small cell lung, salivary gland, pancreatic, sarcoma or thyroid cancer.

In some embodiments, the tumor specimen has undergone fixation. The method of fixation is not limited and may be any method of fixation known in the art. In some embodiments, the tumor specimen is a formalin-fixed paraffin-embedded (FFPE) tumor specimen. The amount of genomic material in the tumor sample is not limited and may be any amount of genomic material disclosed herein. The area of the tumor sample is not limited and may be any area disclosed herein.

In some embodiments, the method further comprises measuring an expression level of RNA transcripts for a fusion protein in the biological sample obtained from the tumor specimen by PCR amplification of the fusion protein breakpoint. In some embodiments, the fusion protein is an NTRK, ALK, ROS1, or RET fusion protein (e.g., as disclosed herein).

In some embodiments, the method further comprises determining if the tumor will be responsive to a checkpoint inhibitor therapy by measuring the expression levels of RNA transcripts of one or more genes in the biological sample obtained from the tumor specimen found to be correlated with efficacy of the checkpoint inhibitor therapy. In some embodiments, the expression of one or more of the RNA transcripts of PD-L1, PD-L2, CD8A, TOP2A, and GZMA are measured.

In some embodiments, the method further comprises determining if the tumor will be responsive to an anti-cancer therapy by measuring the expression levels of RNA transcripts of one or more gene mutations in the biological sample obtained from the tumor specimen associated with efficacy for the anti-cancer therapy.

In some embodiments, the method further comprises determining the tumor mutation burden and/or the microsatellite instability in the biological sample obtained from the tumor specimen.

In some embodiments, the NTRK inhibitor is one or more selected from the group consisting of AG-879, AZ-23, AZD-1480, Belizatinib (TSR011), BMS-754807, Crizotinib, Entrectinib, Foretinib, GW-2580, K252a, Larotrectinib, Midostaurin (PKC412), and PLX7486.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately,” the invention includes an embodiment in which the exact value is recited. For any embodiment of the invention in which a numerical value is not prefaced by “about” or “approximately,” the invention includes an embodiment in which the value is prefaced by “about” or “approximately.”

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated.”

### Examples

In some embodiments, expression levels are measured with primers targeting both the 5′ and 3′ ends of each gene. Imbalance scores capture the ratio of 3′ expression relative to 5′ expression plus a regularization offset. High 3′ expression relative to 5′ expression can be interpreted as evidence that the 3′ end of the gene has been fused to the 5′ end (including the promoter region) of a different gene with higher typical expression levels-particularly when the absolute 3′ expression is also high relative to the normal population. Isoform-specific primers that directly target sequence spanning the junction between hypothesized fusion partners demonstrate sensitivity of the method for NTRK1 (e.g., ATP1B1-NTRK1) and NTRK3 (e.g., PML-NTRK3, KANK1-NKTRK3).

### ROS1 p.G2032R

ROS1 p.G2032R mutations are recurrent, and the most common resistance mutation observed in patients with ROS1 rearranged lung cancer treated with crizotinib¹. The glycine-to-arginine substitution at codon 2032 in the ROS1 kinase domain occurs in the solvent front in the distal end of the kinase hinge and ATP-binding site of ROS1, which is believed to result in steric hindrance with the piperidine ring of crizotinib². While ROS1 p.G2032 mutation renders crizotinib treatment ineffective for patients expressing this mutation, favorable responses have been reported in patients possessing this mutation treated with next generation inhibitors lorlatinib and repotrectinib^{3,4}. In vitro and in-vivo sensitivity to cabozantinib and DS-6051b in the same patient cohort have also been reported^{5,6}. ¹ Gainor, Justin F et al. “Patterns of Metastatic Spread and Mechanisms of Resistance to Crizotinib in ROS1-Positive Non-Small-Cell Lung Cancer.” JCO precision oncology vol. 2017 (2017): PO.17.00063. doi:10.1200/PO.17.00063² Awad, Mark M et al. “Acquired resistance to crizotinib from a mutation in CD74-ROS1.” The New England journal of medicine vol. 368,25 (2013): 2395-401. doi: 10.1056/NEJMoa1215530³ Annals of Oncology, Volume 29, Supplement 8, 2018, Pages viii493-viii547, ISSN 0923-7534, https://doi.org/10.1093/annonc/mdy292.045.⁴ Current, E. S. M. O. “Highlights in respiratory oncology.”⁵ Katayama, Ryohei et al. “The new-generation selective ROS1/NTRK inhibitor DS-6051b overcomes crizotinib resistant ROS1-G2032R mutation in preclinical models.” Nature communications vol. 10,1 3604, 9 Aug. 2019, doi: 10.1038/s41467-019-11496-z⁶ Katayama, Ryohei et al. “Cabozantinib overcomes crizotinib resistance in ROS1 fusion-positive cancer.” Clinical cancer research: an official journal of the American Association for Cancer Research vol. 21,1 (2015): 166-74. doi: 10.1158/1078-0432.CCR-14-1385

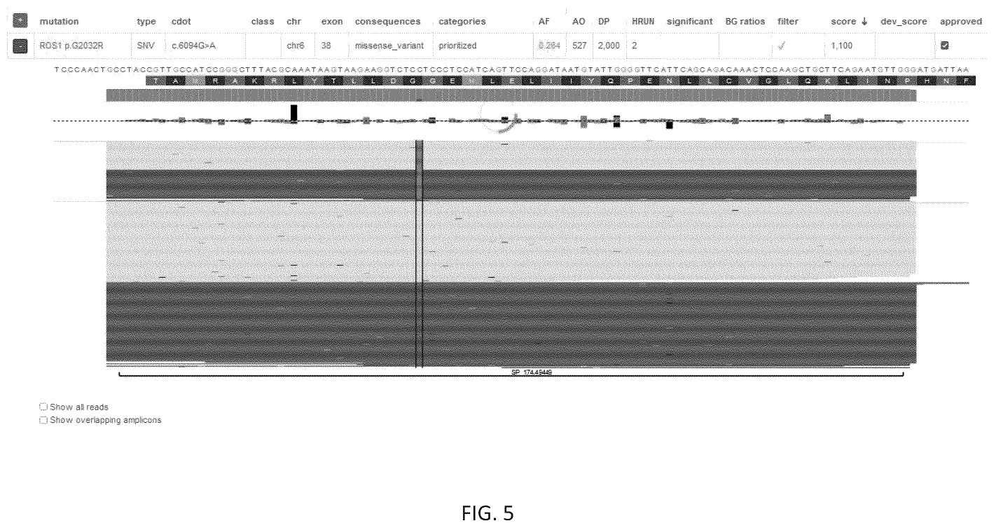

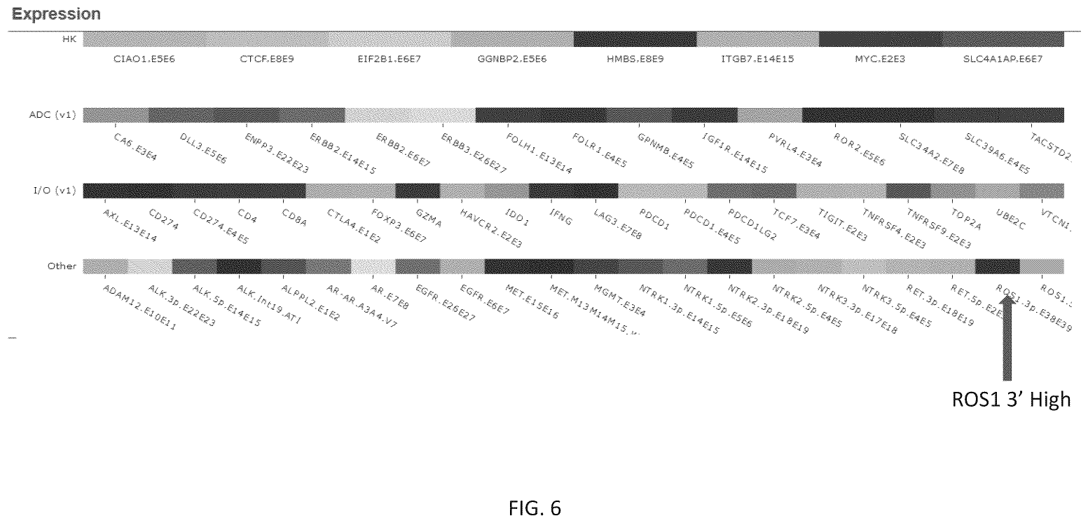

To further demonstrate the methods encompassed by this invention, next-generation sequencing was performed on a tissue sample using Applicant's StrataNGS®, a multiplex PCR, semiconductor sequencing-based comprehensive genomic profiling test. The results did not identify any reportable alterations for ALK fusion, BRAF hotspot, EGFR hotspot, ERBB2 hotspot, MET amplification, MET fusion, RET fusion or a ROS1 fusion. The results also revealed microsatellite stability, low PD-L1 expression and the sample was assessed as having a low Strata Immune Signature. Nonetheless, the sequence read information of ROS1 depicted in FIG. 5, does demonstrate the tissue contains a ROS1 p.G2032R mutation, with an estimated variant allele frequency of 26%. To further elucidate whether the tissue contains a ROS1-5′ partner fusion not targeted by StrataNGS, expression levels of ROS1 3′ exon and ROS1 5′ exon were determined and then normalized against at least the housekeeping genes CIAO1.E5E6, EIF2B1.E6E7 and HMBS.E8E9. As depicted in FIG. 6, ROS1 3′ exon shows a high level of expression, whereas the ROS1 5′ exon does not show this same high level of expression. This mismatch of ROS1 3′ exon expression to ROS1 5′ exon expression is indicative of the presence of a ROS1 fusion protein, particularly a ROS1 fusion with a 5′ partner not targeted by StrataNGS®. Thus, these results demonstrate the importance of the disclosed method in identifying unknown ROS1 fusions, which previous comprehensive genomic profiling diagnostic tools were incapable of identifying.

### ALK p.G1202R and ALK p.G1269A

Anaplastic lymphoma kinase (ALK) rearrangements can result in expression of ALK fusion proteins which are constitutively active. Treatment of this small proportion of ALK positive non-small cell lung cancer patients involve the administration of ALK inhibitors, such as first generation ALK-inhibitor crizotinib. Yet frequently, successful treatment gives way to an ALK gene mutation driven resistance, requiring the administration of second generation ALK inhibitors, such as ceritinib, alectinib and brigatinib. One of the most prevalent ALK mutations that imparts resistance to the second generation ALK inhibitors is the solvent front ALK G1202R substitution⁷. Another such ALK mutation identified in a patient with resistance to second generation ALK inhibitors is the ALK G1269A substitution. Recently, a patient resistant to the third generation ALK inhibitor lorlatinib was demonstrated to have both the ALK G1202R substitution as well as the ALK G1269A substitution⁷, thus underscoring the importance of identifying ALK mutations within a tumor sample as well as the presence of ALK fusions which are constitutively active and thus represent potent oncogenic drivers. ⁷ Yoda, Satoshi et al. “Sequential ALK Inhibitors Can Select for Lorlatinib-Resistant Compound ALK Mutations in ALK-Positive Lung Cancer.” Cancer discovery vol. 8,6 (2018): 714-729. doi: 10.1158/2159-8290.CD-17-1256

A tissue sample from a patient with non-small cell lung cancer was assayed using Strata-NGS® as discussed in the first example. The results did not identify reportable genomic alterations in BRAF, EGFR, ERBB2, MET, RET or ROS1, nor a CDKN2A deep deletion. The tissue sample was identified as being microsatellite stable, as having a low tumor mutational burden and as having high PD-L1 expression. The tissue was also identified as having the ALK p.G1202R substitution mutation, with an estimated variant allele frequency of 25% and the ALK p.G1269A mutation, with an estimated variant allele frequency of 24%. While an ALK fusion was not detected by the StrataNGS® test, high expression of the 3′ exon of ALK was observed, suggesting the presence of an ALK fusion with a 5′ partner not targeted by StrataNGS®.

What is claimed is:

1. A method of treatment, comprising:
a. measuring an expression level of RNA transcripts of a gene, a 3′ exon portion of the gene, a 5′ exon portion of the gene, and at least one reference gene, in a biological sample obtained from a tumor specimen from a subject;
b. normalizing the measured expression levels of the RNA transcripts of the gene, the 3′ exon portion of the gene, and the 5′ exon portion of the gene to the expression level of the RNA transcripts of the at least one reference gene;
c. determining that the tumor expresses a fusion protein when
i. the normalized expression level of the RNA transcripts of the gene is greater than a baseline expression level of RNA transcripts of the gene, and
ii. the ratio of the normalized expression level of the RNA transcripts of the 3′ exon portion of the gene to the normalized expression level of the RNA transcripts of the 5′ exon portion of the gene is greater than 4; and
d. administering an inhibitor to the tumor identified as expressing a fusion protein, wherein the gene is selected from the group consisting of NTRK, ROS1, RET and ALK.

2. The method of claim 1, wherein the gene is a NTRK gene.

3. The method of claims 1-2, wherein the NTRK gene is selected from NTRK1, NTRK2, and NTRK3.

4. The method of claim 1, wherein the gene is ROS1.

5. The method of claim 1, wherein the gene is ALK.

6. The method of claim 1, wherein the gene is RET.

7. The method of claims 2-3, wherein the expression level of the RNA transcripts of the NTRK gene is determined by measuring the expression of RNA transcripts of a portion of the NTRK gene comprising the junction of exons 16-17.

8. The method of claims 2-3, wherein measuring an expression level of a 3′ exon portion of the NTRK gene comprises measuring the expression of RNA transcripts of one or more portions of the NTRK gene comprising the junction of exons 14-15, 15-16, 16-17, and/or 17-18.

9. The method of claims 2-3, wherein measuring an expression level of a 5′ exon portion of the NTRK gene further comprises measuring the expression of RNA transcripts of one or more portions of the NTRK gene comprising the junction of exons 2-3, 3-4, and/or 4-5.

10. The method of claims 1-9, wherein the at least one reference gene comprise at least one gene selected from LRP1, MRPL13, TBP, HMBS, ITGB7, MYC, CIAO1, CTCF, EIF2B1, GGNBP2, and SLC4A1AP.

11. The method of claims 1-10, wherein the tumor specimen is a formalin-fixed paraffin-embedded (FFPE) tumor specimen.

12. The method of claims 1-11, wherein the expression levels of RNA transcripts are measured using PCR and next-generation sequencing (NGS).

13. The method of claims 1-12, further comprising measuring an expression level of RNA transcripts for a fusion protein in the biological sample obtained from the tumor specimen by PCR amplification of the fusion protein breakpoint.

14. The method of claim 13, wherein the fusion protein is an NTRK, ALK, ROS1, or RET fusion protein.

15. The method of claims 1-14, further comprising determining if the tumor will be responsive to a checkpoint inhibitor therapy by measuring the expression levels of RNA transcripts of one or more genes in the biological sample obtained from the tumor specimen found to be correlated with efficacy of the checkpoint inhibitor therapy.

16. The method of claims 1-15, further comprising determining if the tumor will be responsive to an anti-cancer therapy by measuring the expression levels of RNA transcripts of one or more gene mutations in the biological sample obtained from the tumor specimen associated with efficacy for the anti-cancer therapy.

17. The method of claims 1-16, further comprising determining the tumor mutation burden and/or the microsatellite instability in the biological sample obtained from the tumor specimen.

18. The method of claims 1-17, wherein the inhibitor is a NTRK inhibitor and is one or more selected from the group consisting of AG-879, AZ-23, AZD-1480, Belizatinib (TSR011), BMS-754807, Crizotinib, Entrectinib, Foretinib, GW-2580, K252a, Larotrectinib, Midostaurin (PKC412), and PLX7486.

19. A method of identifying a subject that would benefit from NTRK inhibitor therapy, comprising
a. measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject;
b. normalizing the measured expression levels of the RNA transcripts of the NTRK gene, the 3′ exon portion of the NTRK gene, and the 5′ exon portion of the NTRK gene to the expression level of the RNA transcripts of the at least one reference gene;
c. determining that the tumor expresses an NTRK fusion protein when
i. the normalized expression level of the RNA transcripts of the NTRK gene is greater than a baseline expression level of RNA transcripts of the NTRK gene, and
ii. the ratio of the normalized expression level of the RNA transcripts of the 3′ exon portion of the NTRK gene to the normalized expression level of the RNA transcripts of the 5′ exon portion of the NTRK gene is greater than 4; and
d. providing a determination if the subject has a tumor expressing an NTRK fusion protein and therefore would benefit from NTRK inhibitor therapy.

20. A method of treatment of a subject in need thereof with a NTRK inhibitor therapy, comprising administering to said subject the NTRK inhibitor therapy, wherein said subject in need thereof was previously identified by a method comprising:
a. measuring an expression level of RNA transcripts of a Neurotrophic Tyrosine Receptor Kinase (NTRK) gene, a 3′ exon portion of the NTRK gene, a 5′ exon portion of the NTRK gene, and at least one reference gene, in a biological sample obtained from a tumor specimen from the subject;
b. normalizing the measured expression levels of the RNA transcripts of the NTRK gene, the 3′ exon portion of the NTRK gene, and the 5′ exon portion of the NTRK gene to the expression level of the RNA transcripts of the at least one reference gene; and
c. determining that the tumor expresses an NTRK fusion protein when
i. the normalized expression level of the RNA transcripts of the NTRK gene is greater than a baseline expression level of RNA transcripts of the NTRK gene, and
d. the ratio of the normalized expression level of the RNA transcripts of the 3′ exon portion of the NTRK gene to the normalized expression level of the RNA transcripts of the 5′ exon portion of the NTRK gene is greater than 4.
